(19) United States
(12) Patent Application Publication
(10) Pub. No.: US 2012/0135070 A1
(43) Pub. Date: May 31, 2012

(54) Copolymers

(75) Inventors: Alexander Kros, Leider (NL); Hana Robson Marsden, Wellington (NZ); Wim John Jesse, Rapenburg (NL)

(73) Assignee: UNIVERSITEIT LEIDEN, Leiden (NL)

(21) Appl. No.: 13/266,732

(22) PCT Filed: Apr. 26, 2010
(86) PCT No.: PCT/EP2010/002547

§ 371 Date: Feb. 14, 2012

(30) Foreign Application Priority Data
Apr. 28, 2009 (GB) 0907251.3

(51) Int. Cl. A61K 39/145; A61P 31/16; A61K 39/395; A61P 37/04; C07K 19/00; A61K 9/48

(52) U.S. Cl. 424/451; 530/350; 424/209.1; 424/134.1

(57) ABSTRACT

The invention provides a block copolypeptide comprising a hydrophilic heteropolypeptide block (A) and a hydrophobic homopolypeptide block (B). There is also provided a polymersome comprising a block copolypeptide of the invention. The invention further provides a method for preparing a copolymer comprising ring-opening polymerisation (ROP) of an amino acid N-carboxyanhydride (NCA) initiated from a peptide.

The invention relates to block copolymers of polypeptides and polymersomes containing such copolymers. The invention also is directed to methods for preparing these block copolymers and polymersomes, and their uses.

### BACKGROUND OF THE INVENTION

This listing or discussion of an apparently prior-published document in this specification should not necessarily be taken as an acknowledgement that the document is part of the state of the art or is common general knowledge.

Polypeptides can be programmed with the ability to adopt specific intra- and intermolecular conformations, which may allow heightened levels of control over the morphologies and properties of the self-assembled structures. The structure and functional properties of proteins and peptides are determined by the primary sequence of amino acids. Materials scientists are still unable to design the primary sequence to have as high a level of control over the three-dimensional folded structure and intermolecular recognition that are present in nature.

There has been some progress however, particularly in understanding the folding of silks, elastins, collagens, and coiled-coil motifs (van Hest, J. C. M.; Tirrell, D. A. Chemical Communications 2001, (19), 1897-1904). Two methods for the synthesis of polypeptides that assemble in a well defined manner are the ring-opening polymerization (ROP) of amino acid N-carboxyanhydrides (NCAs), and solid-phase synthesis.

The ROP of NCAs is the most common method of synthesizing polypeptides containing a single amino acid residue (Smeenk, J. M.; Ayres, L.; Stunnenberg, H. G.; van Hest, J. C. M. Macromolecular Symposia 2005, 225, 1-8). Such polypeptides are also referred to herein as homopolypeptides. These polymers can be readily prepared, and have no detectable racemization at the chiral centers (Deming, T. J., Polypeptide and polypeptide hybrid copolymer synthesis via NCA polymerization. 2006; Vol. 202).

Blocks based on glutamic acid (y-benzyl L-glutamate) have been commonly synthesized as their polymerization is thought to be the best controlled, and because they form well-defined rod-like α-helical secondary structures in the solid-state and solution (Gallot, B. Progress In Polymer Science 1996, 21, (6), 1035-1088). They have been initiated from traditional linear coil polymers, polymer dendrimers (Huang, H.; Dong, C. M.; Wei, Y. Combinatorial Chemistry & High Throughput Screening 2007, 10, (5), 368-376 and Higashi, N.; Koga, T.; Niwa, M. Langmuir 2000, 16, (7), 3482-3486), modified lipids (Dimitrov, I. V.; Berlinova, I. V.; Iliev, P. V.; Vladimirov, N. G. Macromolecules 2008, 41, (3), 1045-1049), and polypeptides themselves synthesized by ROP of NCAs (Sun, J.; Chen, X. S.; Lu, T. C.; Liu, S.; Tian, H. Y.; Guo, Z. P.; Jing, X. B. Langmuir 2008, 24, (18), 10099-10106). The most common initiator is primary amine end-groups, but the polymerization can also be initiated with transition metal-amine functionalized polymers (Brzezinska, K. R.; Deming, T. J. Macromolecules 2001, 34, (13), 4348-4354).

Block copolymers have also been synthesized in the reverse manner, i.e. the ROP of NCA, followed by polymerization of another polymer from the polypeptide (Kros, A.; Jesse, W.; Metselaar, G. A.; Cornelissen, J. J. L. M. Angewandte Chemie-International Edition 2005, 44, (28), 4349-4352 and Imanishi, Y. Journal of Macromolecular Science-Chemistry 1984, A21, (8-9), 1137-1163).

The ROP of NCAs has a disadvantage of multiple side-reactions and termination reactions, resulting in polypeptides with a wide range of polymer lengths. To reduce the range of lengths, which are likely to have different self-assembly properties, inconvenient fractionation is often applied. Additionally the abundance of side-reactions leads to homopolymer contamination, which has to be separated from the block copolymer, (Deming, T. J., Polypeptide and polypeptide hybrid copolymer synthesis via NCA polymerization. 2006; Vol. 202).

In addition to addressing at least some of the foregoing drawbacks with ROP of NCAs, it would be desirable to develop new copolymers of polypeptides which are able to self-assemble into well defined structures. There is also a continuing need to develop new drug delivery devices.

### SUMMARY OF THE INVENTION

The subject invention addresses the foregoing and other needs and deficiencies by the provision of a block copolypeptide comprising a hydrophilic heteropolypeptide block (A) and a hydrophobic homopolypeptide block (B). Unless otherwise stated, this is referred to herein as a block copolypeptide of the invention.

A process for preparing a block copolypeptide of the invention is provided. In a further aspect, the invention provides a method for preparing a copolymer comprising ring-opening polymerisation (ROP) of an amino acid N-carboxyanhydride (NCA) initiated from a peptide. Unless otherwise stated, this is referred to herein as a method of the invention.

In another embodiment, there is provided a polymersome (also referred to herein as a peptosome) comprising a block copolypeptide of the invention. Unless otherwise stated, this is referred to herein as a polymersome of the invention. A process for preparing a polymersome of the invention is also provided.

In an alternative embodiment, there is provided a block copolypeptide of the invention or a polymersome of the invention for use in medicine.

In another aspect, the invention provides a drug delivery device comprising a block copolypeptide of the invention or a polymersome of the invention. In another aspect, there is also provided a block copolypeptide of the invention or a polymersome of the invention for use as a drug delivery device.

In a further embodiment, there is provided (i) a block copolypeptide of the invention or a polymersome of the invention for use as a tool in vaccine development, and (ii) the use of a block copolypeptide of the invention or a polymersome of the invention in the manufacture of a tool for vaccine development.

In an alternative aspect, the invention provides (i) a block copolypeptide of the invention or a polymersome of the invention for use in treating influenza, and (ii) the use of a block copolypeptide of the invention or a polymersome of the invention in the manufacture of a medicament for treating influenza.

### DETAILED DESCRIPTION

The invention provides a block copolypeptide comprising a hydrophilic heteropolypeptide block (A) and a hydrophobic homopolypeptide block (B). For the avoidance of doubt, block (A) is covalently attached to block (B) in the block copolypeptide of the invention.

By the term “hydrophilic heteropolypeptide block (A)”, we include the meaning of a polypeptide containing at least two different amino acid residues, wherein the heteropolypeptide block is more soluble in water or other polar solvents (e.g. protic solvents such as alcohols) than in oil or other hydrophobic solvents (e.g. hydrocarbons). Although referred to herein as a polypeptide, the “hydrophilic heteropolypeptide block (A)” may also be considered to be a hydrophilic peptide block (A) containing at least two different amino acids.

Hydrophilic amino acid residues are generally considered to be Arg (A), Asn (N), Asp (D), Gln (Q), Glu (E), Lys (K), Ser (S) and Thr (T). Hydrophobic residues are generally considered to be Ala (A), Ile (I), Leu (L), Met (M), Phe (F), Trp (W), Tyr (Y) and Val (V). Any sequence of amino acid residues may be used in heteropolypeptide block (A), provided that the block is, overall, hydrophilic in nature. Block (A) may also include any non-natural or modified amino acid having the general structure

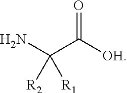

R₁ and/or R₂ may, for example, independently represent a fluorinated side chain (e.g. a fluorinated alkyl group) or a urea derived side chain. One of R₁ or R₂ may be a side chain found in natural amino acids. β amino acids may also be used.

In one embodiment, heteropolypeptide block (A) is a random peptide generated by polymerisation of at least two different amino acids, for example by ROP.

Preferably, however, heteropolypeptide block (A) is not a random peptide generated by polymerisation of at least two different amino acids. Instead, block (A) preferably has a defined amino acid sequence, and thus an exact mass. Such blocks may be prepared by solid phase peptide synthesis (SPPS). Examples of heteropolypeptide blocks (A) with a defined amino acid sequence are set out later in this specification.

In one aspect, the heteropolypeptide block (A) is a helix.

The hydrophilic heteropolypeptide block (A) preferably is capable of forming a coiled coil with a complementary peptide. This feature is thought to be important because it can allow coupling of other molecules to block (A) via a coiled-coil interaction.

Block (A) may be a heteropolypeptide block of any suitable length, preferably wherein it can form a coiled coil with a complementary peptide. The length of block (A), and thus the length of the complementary peptide and the size of the coiled coil, may be designed to fit the use of the block copolypeptide of the invention.

Suitable sequences of amino acid residues that may be used in heteropolypeptide block (A) to form a coiled coil with a complementary peptide are described, for example, in Woolfson, D. N., The design of coiled-coil structures and assemblies, Fibrous Proteins: Coiled-Coils, Collagen And Elastomers, Elsevier Academic Press Inc: San Diego, 2005; Vol. 70, pp 79-112, and in Mason, J. M. et al, Chem Bio Chem, 2004, 5, 170-176, both of which are incorporated herein by reference.

In a preferred aspect, the heteropolypeptide block (A) comprises from 2 to about 200 (e.g. about 3 to about 100, such as from about 3 to about 10, 20, 30 40 or 50) heptads, enabling the block (A) to form a left-handed coiled coil with a complementary peptide.

When block (A) is prepared by solid phase peptide synthesis (SPPS), it may comprise from about 3 to about 10 heptad repeats, e.g. 3, 4, 5, 6, 7, 8, 9 or 10 heptad repeats.

A heptad repeat in block (A) may be denoted (a-b-c-d-e-f-g)ₙ, and (a′-b′-c′-d′-e′-f′-g′)ₙ, using the helical wheel representation, in the complementary peptide. Typically, a and d are non-polar core amino acid residues found at the interface of the block (A) and complementary peptide helices, and e and g are solvent exposed, polar amino acid residues. Using this nomenclature, each heptad may start with any of a, b, c, d, e, f or g (or a′, b′, c′, d′, e′, f′ or g′), not necessarily a or a′. For example, the heptad repeat may be denoted (g-a-b-c-d-e-f)ₙ.

Two or more of the heptads in Block (A) may contain the same repeating sequence of seven amino acids. Alternatively, each heptad in Block (A) may be the same or each may be different.

In an embodiment, each heptad repeat in block (A) may be (E I A A L E K). Thus, block (A) may be (E I A A L E K)ₙ, preferably wherein n is from about 3 to about 10. For example, when n=3, block (A) may be Ac-G(E I A A L E K)₃—NH₂, also known as the peptide E (Marsden, H. R.; Korobko, A. V.; van Leeuwen, E. N. M.; Pouget, E. M.; Veen, S. J.; Sommerdijk, N. A. J. M.; Kros, A. Journal of the American Chemical Society 2008, 130, (29), 9386-9393, incorporated herein by reference).

In a further embodiment, each heptad repeat in block (A) may be (K I A A L K E). Thus, block (A) may be (K I A A L K E)ₙ wherein n is from about 3 to about 10. For example, when n=3, the complementary peptide may be Ac-G(K I A A L K E)₃—NH₂, also known as the peptide K.

In an alternative aspect of the block copolypeptide of the invention, the heteropolypeptide block (A) comprises from 2 to about 200 (e.g. about 3 to about 100, such as from about 3 to about 10, 20, 30 40 or 50) undecatad repeat units, enabling the block (A) to form a right-handed coiled coil with a complementary peptide.

When block (A) is prepared by solid phase peptide synthesis (SPPS), it may comprise from about 3 to about 10 or from about 3 to about 7 undecatad repeats, e.g. 3, 4, 5, 6, 7, 8, 9 or 10 heptad repeats.

The block copolypeptide of the invention contains a hydrophobic homopolypeptide block (B). By the term hydrophobic homopolypeptide block, we include:
(i) any homopolyamino acid wherein the amino acid is hydrophobic, such as alanine (A), leucine (L), isoleucine (I), methionine (M), phenylalanine (F), tryptophan (W), tyrosine (Y) and valine (V), for instance V, L and A; or
(ii) any homopolyamino acid wherein the amino acid is hydrophilic, but where the polar group is protected to render the polyamino acid hydrophobic. Typical hydrophilic (also denoted “polar” in the art) amino acids include arginine (R), asparagine (N), aspartic acid (D), glutamine (Q), glutamic acid (E), histidine (H), lysine (K), serine (S) and threonine (T). Examples of homopolyamino acids wherein the amino acid is hydrophilic, but where the polar group of the amino acid is protected by a hydrophobic protecting group to render it hydrophobic, include poly(benzyl lysine) and poly(benzyl glutamate)(PBLG); or
(iii) any homopolyamino acid wherein the amino acid is a non-natural or modified amino acid having the general structure

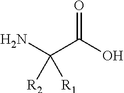

as described hereinbefore.

In any case, the homopolypeptide block (B) typically is more soluble in oil or other hydrophobic solvents (e.g. hydrocarbons) than in water or other polar solvents (e.g. protic solvents such as alcohols).

The hydrophobic homopolypeptide block (B) typically includes from about 10 to about 1000 amino acid residues, preferably from about 10 to about 500 or about 15 to about 400, for example from about 20 to about 300.

In one embodiment, the hydrophobic homopolypeptide block (B) is capable of self-assembling into a three-dimensional configuration. By the term three-dimensional configuration, we include any configuration formed by non-covalent interactions (e.g. van der waals forces or hydrogen bonds) between amino acid residues. Examples of such configurations include α-helices, β-sheets, 3₁₀-helices, π-helices, turns, β-bridges and bends.

For instance, PBLG, which is a preferred hydrophobic homopolypeptide block (B), may form either α-helices and β-sheets, depending on its chain length. PBLG α-helices typically form when there are about 10 or more BLG monomers in the copolymer chain. PBLG β-sheets typically form when there are from about 2 to about 10 BLG monomers in the copolymer chain.

In one aspect, PBLG α-helices are preferred as the hydrophobic homopolypeptide block (B). Typically, these contain from about from about 10 to about 500 or about 15 to about 400, for example from about 20 to about 300 PBLG monomers.

The invention provides a process for preparing the block copolypeptide of the invention comprising the steps of:
(a) preparing a hydrophilic heteropolypeptide block (A);
(b) preparing a hydrophobic homopolypeptide block (B); and
(c) covalently attaching block (A) to block (B) to form the block copolypeptide.

Any suitable method for preparing the heteropolypeptide block (A) may be used. For example, when block (A) has a specific sequence of amino acids (a designed heteropolypeptide), it can be synthesised manually, by SPPS, or by genetically modifying an organism to express it. Random heteropolypeptides can also be synthesised by ROP of NCAs.

Advantageously, step (a) comprises solid phase peptide synthesis (SPPS) of the heteropolypeptide block (A) (Synthetic peptides: a user's guide, Gregory A. Grant, Edition 2, Oxford University Press US, 2002, which is herein incorporated by reference). Using SPPS, the heteropolypeptide block (A) can be designed to have not only a well defined shape (as is possible with NCA derived polypeptides), but also monodisperse size, and additionally have well defined and more complex functionality.

Any suitable method for preparing the homopolypeptide block (B) may be used. For example, block (B) can be synthesised manually, by SPPS, by genetically modifying an organism to express it, or by ring-opening polymerisation (ROP) of an amino acid N-carboxyanhydride (NCA) to form the homopolypeptide block (B). Preferably, block (B) is prepared by ROP of an NCA.

Steps (a), (b) and (c) of the process of the invention may be carried out in any order, and/or simultaneously.

In one aspect, step (a) is carried out before steps (b) and (c). Steps (b) and (c) may be carried out simultaneously.

Alternatively, step (b) may be carried out before steps (a) and (c). Steps (a) and (c) may be carried out simultaneously. For instance, block (B) may be prepared by ROP of an NCA (optionally initiated from a resin), following by SPPS to make block (A).

In a currently preferred embodiment, block (A) is prepared in step (a) by SPPS. ROP of an NCA is initiated from the heteropolypeptide block (A) to produce block (B) and, accordingly, the block copolypeptide of the invention. Thus, step (c) is carried out simultaneously with step (b) (and after step (a)).

In a preferred aspect, the amine terminus of the heteropolypeptide block (A), while block (A) is still anchored to the resin used in its solid phase synthesis, may be use to initiate the ROP of the NCA to form the homopolypeptide block (B), thereby simultaneously covalently attaching block (A) to block (B) to form the block copolypeptide.

The above process gives access to block copolypeptides of the invention with well-defined block sizes and functionalities. Additionally, it overcomes one of the main disadvantages of NCA polymerization, as any block (B) homopolymer that forms can be readily washed away from the resin.

Accordingly, in another embodiment, the invention provides a method for preparing a copolymer comprising ring-opening polymerisation (ROP) of an amino acid N-carboxyanhydride (NCA) initiated from a peptide.

In one aspect, this method comprises solid phase synthesis of the peptide, preferably wherein ROP of the NCA is initiated from the (N-terminus of the) peptide on a solid support.

In an embodiment, the invention provides a polymersome comprising a block copolypeptide.

Preferably, the polymersome comprises a block copolypeptide and a complementary peptide.

The polymersome (or peptosome) may be described as a non-covalent complex of the block copolypeptide, and optionally the complementary peptide.

The complementary peptide typically comprises any peptide capable of forming a coiled coil with the hydrophilic heteropolypeptide block (A) of the block copolypeptide of the invention. The complementary peptide suitably comprises a heteropolypeptide block having a length, enabling it to form a coiled coil with block (A). The length of block (A) and the complementary peptide, and thus the size of the coiled coil, may be designed to fit the use of the block copolypeptide/polymersome of the invention.

Suitable sequences of amino acid residues that may be used in the complementary peptide to form a coiled coil with the heteropolypeptide block (A) are described, for example, in Woolfson, D. N., The design of coiled-coil structures and assemblies, Fibrous Proteins: Coiled-Coils, Collagen And Elastomers, Elsevier Academic Press Inc: San Diego, 2005; Vol. 70, pp 79-112, and in Mason, J. M. et al, Chem Bio Chem, 2004, 5, 170-176, both of which are incorporated herein by reference.

In one aspect, the complementary peptide comprises from 2 to about 200 (e.g. about 3 to about 100, such as from about 3 to about 10, 20, 30 40 or 50) heptads, preferably, 3, 4, 5, 6, 7, 8, 9 or 10 heptads. This enables the block (A) to form a left-handed coiled coil with a complementary peptide. The heptad repeat in block (A) may be denoted (a-b-c-d-e-f-g)ₙ, and (a′-b′-c′-d′-e′-′f-g′)ₙ in the complementary peptide. Typically, a and d typically are non-polar core amino acid residues found at the interface of the block (A) and complementary peptide helices, and e and g are solvent exposed, polar amino acid residues.

Two or more of the heptads in the complementary peptide may contain the same repeating sequence of seven amino acids. Alternatively, each heptad in the complementary peptide may be the same or each may be different.

In an embodiment, each heptad repeat in the complementary peptide may be (K I A A L K E). Thus, the complementary peptide may be (K I A A L K E)ₙ wherein n is from about 3 to about 10. For example, when n=3, the complementary peptide may be Ac-G(K I A A L K E)₃—NH₂, also known as the peptide K (Marsden, H. R.; Korobko, A. V.; van Leeuwen, E. N. M.; Pouget, E. M.; Veen, S. J.; Sommerdijk, N. A. J. M.; Kros, A. Journal of the American Chemical Society 2008, 130, (29), 9386-9393, which is incorporated by reference herein).

In a further embodiment, each heptad repeat in the complementary peptide may be (E I A A L E K). Thus, the complementary peptide may be (E I A A L E K)ₙ, preferably wherein n is from about 3 to about 10. For example, when n=3, block (A) may be Ac-G(E I A A L E K)₃-NH₂, also known as the peptide E.

In an alternative aspects of the polymersome of the invention, the complementary peptide comprises from 2 to about 200 (e.g. about 3 to about 100, such as from about 3 to about 10, 20, 30 40 or 50) undecatad repeat units, enabling the complementary peptide to form a right-handed coiled coil with the hydrophilic heteropolypeptide block (A) of the block copolypeptide of the invention.

When the complementary peptide is prepared by SPPS, it typically comprises somewhat less undecatad repeats, such as from about 3 to about 10 or from about 3 to about 7 undecatad repeats, e.g. 3, 4, 5, 6, 7, 8, 9 or 10 heptad repeats.

Any suitable method for preparing the complementary peptide may be used. For example, when the complementary peptide has a specific sequence of amino acids (a designed heteropolypeptide), it can be synthesised manually, by SPPS, or by genetically modifying an organism to express it. Random heteropolypeptides can also be synthesised by ROP of NCAs. Advantageously, the complementary peptide is prepared by SPPS.

The polymersomes of the invention have been shown to encapsulate water soluble compounds (see the Examples). Hence there is potential for use of these materials as drug delivery devices.

The complementary peptide may further comprise a functional group. Any suitable functional group may be used with (e.g. (covalently) attached to) the complementary peptide including, for example, a polymer, copolymer or block copolymer, a ligand, a pharmaceutical agent, a pharmaceutical agent carrier, a fluorescent marker, an antibody, or combination of the foregoing. Thus, through coiled coil formation between block (A) and the complementary peptide, the outside of the polymersomes can be functionalised with targeting/stealth/carrier molecules.

For instance, the complementary peptide may be covalently attached to any water soluble polymer to form a hybrid molecule. Examples of water soluble polymers include poly(ethylene glycol) (PEG). A suitable PEG block may have a chain length of from about 2 to about 200. An example of such a hybrid molecule is the peptide K-PEG hybrid described in Marsden, H. R., et al, Journal of the American Chemical Society 2008, 130, (29), 9386-9393.

The invention provides a process for preparing a polymersome of the invention comprising mixing the block copolypeptide (and any complementary peptide present) in a suitable solvent. Suitable solvents include water, phosphate buffered saline (PBS), and any other aqueous buffers such as TAPS, Bicine, Tris, Tricine, HEPES, TES, MOPS, PIPES, Cacodylate and MES.

Known methods for preparing polymersomes may be used in the above process, including film hydration, solvent injection and sonication (Kita-Tokarczyk, K.; Grumelard, J; Haefele, T.; Meier, W. Polymer 2005, 46 (11) 3540-3563, which is incorporated by reference herein). Sonication currently is a preferred method.

### EXAMPLES

The invention will now be described in detail with reference to particular block copolypeptides and polymersomes of the invention, and processes for making them. For the avoidance of doubt it is to be understood that the information in the Examples is non-limiting. Moreover, any of the features described in the Examples may be combined, as appropriate, with any of the features of the invention set out in the description hereinbefore.

Synthesis and Characterization of Protected PBLG-E Block Copolymer Series.

Poly(α-amino acid)s can be prepared by ring opening polymerization (ROP) of NCAs starting from nucleophilic attack of the C₅ carbonyl group of the NCA by an initiator such as amines, alkoxide anions, alcohols, transitions metals, and water (Blout, E. R.; Karlson, R. H. Journal of the American Chemical Society 1956, 78, (5), 941-946, which is incorporated by reference herein). In this case, the coiled-coil peptide block E (Table 1) was synthesised on resin using a standard fluorenylmethyloxycarbonyl (Fmoc) solid-phase peptide protocol, and removed the N-terminal Fmoc group.

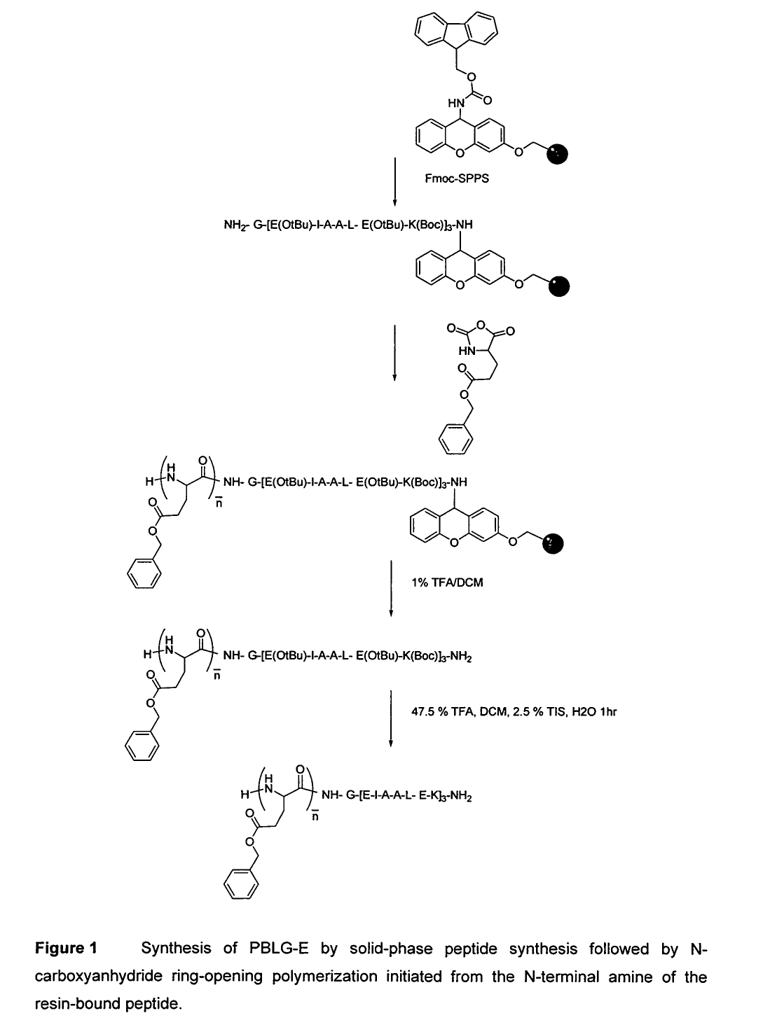

Following this the ROP was initiated by the N-terminal amine of E that was still anchored to the resin (FIG. 1). The polymerization was conducted by shaking the resin-bound peptide with the NCA in DCM at room temperature under an argon atmosphere for one to three days. When the reaction of NCA monomer was complete the resin was drained and washed thoroughly with DCM, NMP, and DMF. It was found that typically 8% of the NCA monomer formed short oligomers during the polymerization reaction, as evidenced by GPC. This is because trace amounts of poorer nucleophiles such as water in the reaction vessel react with the monomer.

An advantage of conducting ROP initiated from a solid-support is that any poly(α-amino acid) that forms in solution during the polymerization can be rinsed away before releasing the block copolymer from the resin. This eases the purification, which was achieved by precipitation of molecules with hydrophobic character in methanol.

The protected peptide block copolymers were released from the solid support by shaking 10 times (2 minutes each) in 99:1 (v/v) DCM:TFA, with subsequent precipitation in cold methanol. The purity of each fraction was ascertained with GPC, from which it was found that within each synthesis the longer PBLG-E hybrids were cleaved first from the resin, with a progressive shortening of the PBLG chain with each fraction collected, until finally peptide fragments from the solid-phase peptide synthesis of E were cleaved.

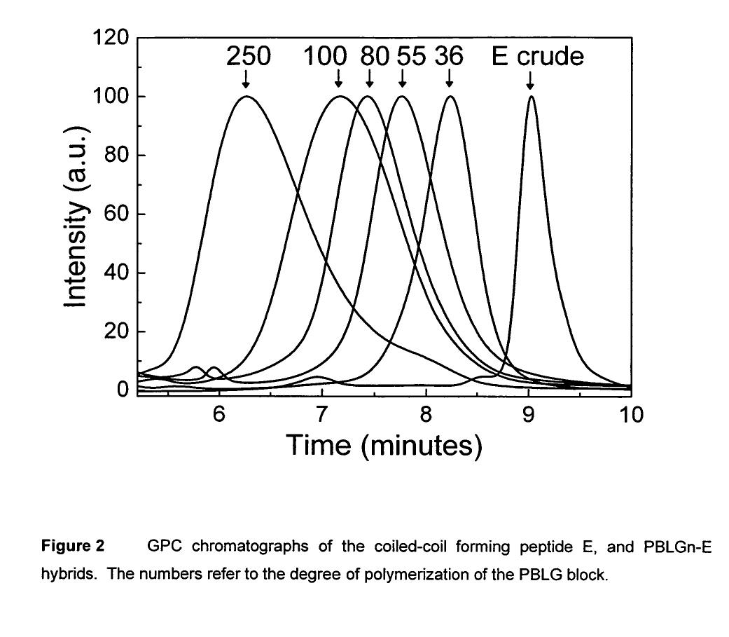

In this way peptide block copolymers with a lower polydispersity index (PDI) can be obtained by selecting which fractions to combine. Due to the washing away of homo-PBLG while the block copolymer is still attached to the resin, and the cleavage of peptide fragments from the resin only after the bulk of PBLG-E molecules have been cleaved, no further purification was necessary. HPLC analysis of the protected form of PBLG-E revealed that the PBLG-E eluted from the column at ˜80% DCM in one peak, further corroborating the purity and low polydispersity of the hybrid. The GPC chromatographs of the PBLG-E series are shown in FIG. 2. Peaks are monomodal and the PDIs range from 1.1 for the hybrid with the shortest PBLG block to 1.7 for the hybrid with the longest PBLG block.

Synthesis and Characterization of a PBLG-E Block Copolymer Series.

The protecting groups from the glutamic acid and lysine residues of peptide E were removed (by stirring the hybrid PBLG-E in 47.5:47.5:2.5:2.5 (v/v) TFA:DCM:water:TIS for 1 hour), while retaining the benzyl protecting groups of the PBLG block, and the hybrid was precipitated in cold methanol. The complete removal of the protecting groups was confirmed by the disappearance of the Ot-Bu and BOC CH₃ peaks at 1.5 ppm from ¹H NMR spectra.

To determine the degree of polymerization of the PBLG blocks, spectra were obtained for each compound in deuterated dichloromethane with increasing amounts of trifluoroacetic acid, ensuring that there was no aggregation of the amphiphilic block copolymer and hence accurate peak comparisons between E and PBLG blocks could be made (Higashi, N.; Kawahara, J.; Niwa, M. Journal of Colloid and Interface Science 2005, 288, (1), 83-87, and Bradbury, E. M.; Cranerob, C.; Goldman, H.; Rattle, H. W. E. Nature 1968, 217, (5131), 812, both of which are incorporated by reference herein). Note that when PBLG-E is in the α-helical conformation, e.g. in DCM or DMSO, the α-H resonance peak is at 4.0 ppm, and by adding TFA the peak position is shifted low-field to 4.7 ppm, indicating that the hybrids have random coil conformation in this solvent mixture, and are not aggregated.

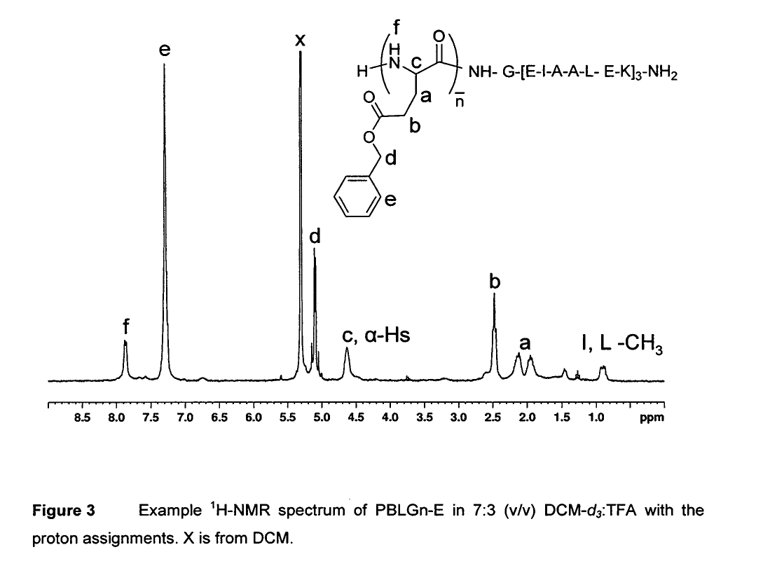

The peak arising from the leucine and isoleucine methyl protons of the E block was compared to the peak arising from the benzyl protons of the PBLG block (FIG. 3). The degree of polymerization of the PBLG blocks as established by ¹⁻H NMR spectroscopy was close to that determined by GPC, indicating that the polystyrene standards used for GPC molecular weight comparison are reliable for these hybrids.

The molecular characteristics of the compounds used in this study are shown in Table 1. This Table includes two examples of PBLG-K block copolypeptides of the invention, which may be prepared using analogous methods to those described in detail herein in relation to the PBLG-E block copolypeptides.

TABLE 1
Molecular Characteristics of the Compounds used in this Study

| | | | MN | |
|---|---|---|---|---|
| name | structure | Yield (%) | (g/mol) | PDI³ |
| K | Ac-(K I A A L K E)₃G-NH₂ | ~40 | 2378.0¹ | |
| E | Ac-G(E I A A L E K)₃-NH₂ | ~40 | 2380.6¹ | |
| K-PEG | Ac-(K I A A L K E)₃G-PEG₇₇ | ~10 | 5832^{1,2} | 1.05¹ |
| PBLG₃₆-E | PBLG₃₆-G(E I A A L E K)₃-NH₂ | 28 | 10230^{2,3} | 1.1 |
| PBLG₅₅-E | PBLG₅₅-G(E I A A L E K)₃-NH₂ | 30 | 14396^{2,3} | 1.3 |
| PBLG₈₀-E | PBLG₈₀-G(E I A A L E K)₃-NH₂ | 56 | 19877^{2,3} | 1.4 |
| PBLG₁₀₀-E | PBLG₁₀₀-G(E I A A L E K)₃-NH₂ | 69 | 24262^{2,3} | 1.4 |
| PBLG₂₅₀-E | PBLG₂₅₀-G(E I A A L E K)₃-NH₂ | 74 | 57148^{2,3} | 1.7 |
| PBLG₃₅-K | PBLG₃₇-G(K I A A L K E)₃-NH₂ | 30 | 10135^{2,3} | 1.3 |
| PBLG₅₀-K | PBLG₅₀-G(K I A A L K E)₃-NH₂ | 35 | 13279^{2,3} | 1.5 |

¹Obtained from MALDI-TOF MS.
²Based on a comparison of ¹H-NMR peaks.
³Fitting GPC traces with polystyrene standards

The hydrophilic peptide E had 22 amino acid residues, while the hydrophobic PBLG block ranges from 36 to 250 benzyl glutamate residues. MALDI-TOF MS was possible for the shortest PBLG-E hybrids. The mass did not correspond to an integer multiple of benzyl glutamate monomers in the PBLG chain. Additionally, the Kaiser test, which is sensitive to amines, was negative. These results indicate that the polymer chains do not end in a primary amine, as would be expected by the “amine” mechanism of ring opening polymerization, but that another reaction, such as the “activated monomer” mechanism, has capped the growing chains. This is also consistent with the fact that there is not 100% monomer conversion, but some degree of oligomer formation. A given polymerization can alternate between these two mechanisms, and ROPs of NCAs using amines as initiators are known for their variable chain-end functionality and formation of homopolymer (Deming, T. J., Polypeptide and polypeptide hybrid copolymer synthesis via NCA polymerization. 2006; Vol. 202, and Klok, H. A. Angewandte Chemie-International Edition 2002, 41, (9), 1509-1513, which are both incorporated by reference herein).

The amide | and amide ∥ positions in FT-IR spectra (1651.1 cm⁻¹ and 1546.9 cm⁻¹ respectively) indicate that PBLG-E adopts a typical α-helical structure in the solid state. There was no shoulder on the amide | vibration, indicating that there was no random coil secondary structure in the hybrid, and illustrating that the secondary structure of E was stable when conjugated with PBLG. The half width at half maximum (HWHM) of the amide ∥ absorption depends on the stability of the α-helix, and at ˜14 cm^{‘1} for the amide ∥ band, this is on a par with the most stable helices (Nevskaya, N. A.; Chirgadze, Y. N. Biopolymers 1976, 15, (4), 637-648, which is incorporated by reference herein).

Geometries of the Molecular Building-Blocks PBLG, E, K and K-PEG

PBLG is hydrophobic and with n larger than 10 has an α-helical secondary structure (Rinaudo, M.; Domard, A. Biopolymers 1976, 15, (11), 2185-2199, which is incorporated by reference herein), resulting in a rod-like molecular shape. The length of PBLG α-helices is n×1.5 nm (Murthy, N. S.; Knox, J. R.; Samulski, E. T. Journal Of Chemical Physics 1976, 65, (11), 4835-4839, which is incorporated by reference herein) hence the PBLG rod-like blocks in this study range in length from 5.4 to 37.5 nm long, and have a diameter of ˜2 nm (Chang, Y. C.; Frank, C. W. Langmuir 1996, 12, (24), 5824-5829, which is incorporated by reference herein).

The peptide E was chosen as the hydrophilic block because it forms an α-helical coiled-coil dimer with K, a peptide with a complementary amino acid sequence. E/K is one of the shortest pairs of coiled-coil forming peptides that specifically forms heterodimers. The secondary and quaternary structures of the peptides E and K in buffered solution were evaluated by circular dichroism spectroscopy. Peptide E adopts a predominantly random coil conformation, while K exhibits a predominantly α-helical spectrum. Both peptides are in the monomeric state as indicated by the observed ellipticity ratios ([θ]222/[θ]208) of 0.59 and 0.74 respectively. When peptides E and K were combined in an equimolar ratio, denoted E/K, a typical α-helical spectrum is exhibited, with minima at 208 nm and 222 nm. The ellipticity ratio was determined to be 1.00, consistent with interacting α-helices. This clearly shows that E and K specifically interact to form a heterodimeric α-helical coiled-coil. The formation of the dimeric species was confirmed by determining the molecular weights using sedimentation equilibrium, revealing that separate solutions of E and K are purely monomeric while the mixture of E/K exists as dimers.

E and K form complexes with a well defined rod-like geometry of cylinders 3.5 nm long with approximately the same diameter as PBLG rods (Lindhout, D. A.; Litowski, J. R.; Mercier, P.; Hodges, R. S.; Sykes, B. D. Biopolymers 2004, 75, (5), 367-375, which is incorporated by reference herein). Poly(ethylene glycol) is a hydrophilic coil polymer, and the PEG used herein, with an average of 77 monomers, has a diameter of approximately 5 nm (the hydrodynamic diameter of the PEG block was determine by DLS). The peptides K and the hybrid K-PEG are predominantly hydrophilic and do not aggregate in aqueous solutions.

The inventors surprisingly have found that these molecules may be used as modular building-blocks for the bottom-up fabrication of nanostructures. In particular, as described herein, by combining equimolar amounts of PBLG-E and K or K-PEG, amphiphilic non-covalent diblock (denoted PBLG-E/K) or triblock (denoted PBLG-E/K-PEG) copolymers were formed. This provides a simple method of adjusting the physical, chemical, and biological properties of the block copolymers.

For clarity and simplicity, these examples describe the synthesis and properties of polymersomes of the invention containing PBLG-E block copolypeptides. Of course, other polymersomes of the invention, such as those containing PBLG-K block copolypeptides (e.g. PBLG-K/E), may also be prepared using analogous methods to those described in detail herein in relation to the PBLG-E block copolypeptides.

Self-Assembling Properties of the Hybrids in Solution.

Due to the amphiphilic nature of the rod-rod hybrids PBLGn-E and the non-covalent complexes PBLGn-E/K and PBLGn-E/K-PEG, the PBLG and hydrophilic blocks were expected to phase separate in aqueous solution. The self assembling characteristics of the PBLG-E series, both in isolation and with equimolar amounts of K or K-PEG were studied in phosphate buffered saline solution (PBS) at pH 7.0. The PBLG-E hybrids, having large hydrophobic PBLG blocks, are not directly soluble in aqueous solutions. The standard methods for polymersome preparation, namely film hydration, solvent injection, and sonication were tested. The most ordered self-assembly was achieved by dissolving the molecules in tetrahydrofuran (THF), which is a common solvent for all of the blocks, and exchanging this for PBS, which is selective for the hydrophilic E, E/K, and E/K-PEG blocks by sonication for two hours in an open vessel. Due to the initial mobility of the molecules in the common solvent, and the high energy input of sonication, the structures that formed were equilibrium structures. When the sonication was stopped the PBLG blocks were immobile and the structures were in frozen equilibrium.

Effect of THF on E/K and PBLG Secondary and Quaternary Structures.

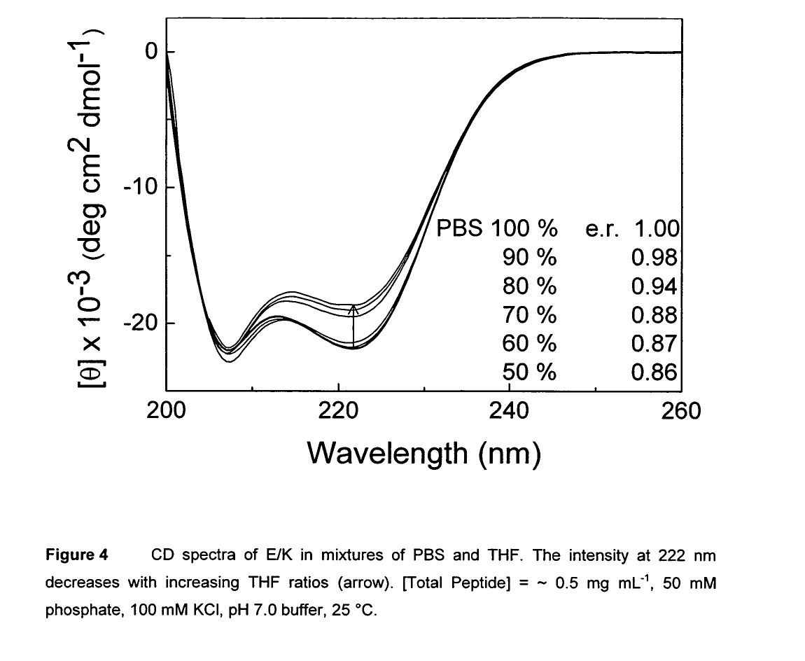

The E/K heterodimer is a non-covalent complex driven by the packing of leucine and isoleucine residues forming a hydrophobic core in order to reduce contact with the aqueous environment. In PBS E/K exhibited a typical α-helical CD spectrum, with minima at 208 nm and 222 nm (FIG. 4). The ellipticity ratio was 1, consistent with interacting α-helices (Zhou, N. E.; Kay, C. M.; Hodges, R. S. Journal of Biological Chemistry 1992, 267, (4), 2664-2670, which is incorporated by reference herein). Upon the addition of THF, the secondary structure of the peptides remained α-helical, but the intermolecular interaction is disrupted, as evidenced by the decreasing elipiticity ratio (FIG. 4). This is thought to be because adding THF to PBS reduces the polarity of the solvent so there is a decreased energetic penalty associated with the hydrophobic residues being exposed to the solvent. PBLG is α-helical in THF, and aggregates in aqueous solutions.

Based on these observations the amount of THF was fixed at 10 (v/v) % in PBS prior to sonication. This is believed to strike a balance between the necessity to perform experiments in an environment allowing coiled-coil pairing between E and K, and the need for mobility of the hydrophobic PBLG blocks in order to reduce the formation of macro-aggregates (samples were prepared using 5, 10, 20, 30, and 40% THF in PBS. Between 10 and 30% THF the particles had similar appearances, whereas with more THF the particles were larger (DLS) and had a different appearance (negative stained TEM)).

Peptide Structure in the Polypeptide Self-Assembled Structures.

CD spectra of the hybrids and complexes in aqueous buffer after sonication are typical for aggregated α-helices: there was dampening of the spectrum and red-shifting of the ‘222 nm’ minimum (see, for example, Potekhin, S. A.; Melnik, T. N.; Popov, V.; Lanina, N. F.; Vazina, A. A.; Rigler, P.; Verdini, A. S.; Corradin, G.; Kajava, A. V. Chemistry & Biology 2001, 8, (11), 1025-1032, and Pandya, M. J.; Spooner, G. M.; Sunde, M.; Thorpe, J. R.; Rodger, A.; Woolfson, D. N. Biochemistry 2000, 39, (30), 8728-8734, both of which are incorporated herein by reference).

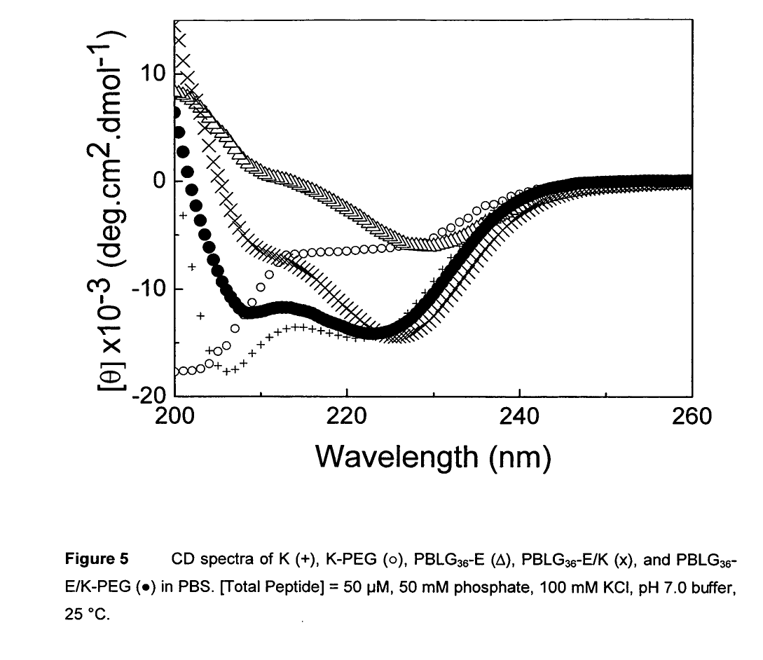

An example of the CD spectra is given in FIG. 5. For PBLG₃₆-E the 222 nm peak was red-shifted, and both peaks were dampened. This is typical for membrane proteins, and the spectral artifacts are attributed to the particulate nature of the suspension (Long, M. M.; Urry, D. W.; Stoeckenius, W. Biochemical and Biophysical Research Communications 1977, 75, (3), 725-731, which is incorporated by reference herein). For soluble proteins and peptides the intensity at 222 nm is directly proportional to the amount of helical structure (Chen, Y. H.; Yang, J. T.; Chau, K. H. Biochemistry 1974, 13, (16), 3350-3359, which is incorporated by reference herein), but in this case the spectra are distorted due to the tight packing and the amount of helical structure cannot be determined.

Upon combining K with PBLG₃₆-E (PBLG₃₆-E/K) the distortions in the spectrum were reduced. With the addition of K-PEG (PBLG₃₆-E/K-PEG), the position of the minima is only slightly red-shifted (223 nm), and there is less dampening of the CD signal. These results show that the longer the hydrophilic block is in comparison to the hydrophobic PBLG block the fewer artifacts present in the CD spectra. Although the E/K pairing can not be directly observed due to juxtaposition of the spectra of E/K with that of PBLG, it is clear that the molecules interact as the spectra differ strongly from the average of the individual components.

Particle Sizes: Dynamic Light Scattering (DLS)

The ability of the PBLG-E molecules and complexes to form well defined structures, and the sizes of these particles, were investigated with DLS. The hybrid with the longest hydrophobic block, PBLG₂₅₀-E, required association with K-PEG in order to have a large enough corona to self-assemble in an ordered manner. For PLBG₁₀₀-E, with a shorter hydrophobic block, the increase in corona size afforded by association with K was sufficient to lead to ordered structures. When the PBLG block length was 80 monomers or shorter the PBLG-E hybrids had a suitable balance of hydrophobicity and hydrophilicity to form ordered self-assembled structures. The average particle sizes ranged from 100 nm to 400 nm, and were significantly larger than the calculated sizes of spherical micelles. All size distributions were monomodal and the polydispersity index of the samples was 0.35 or less.

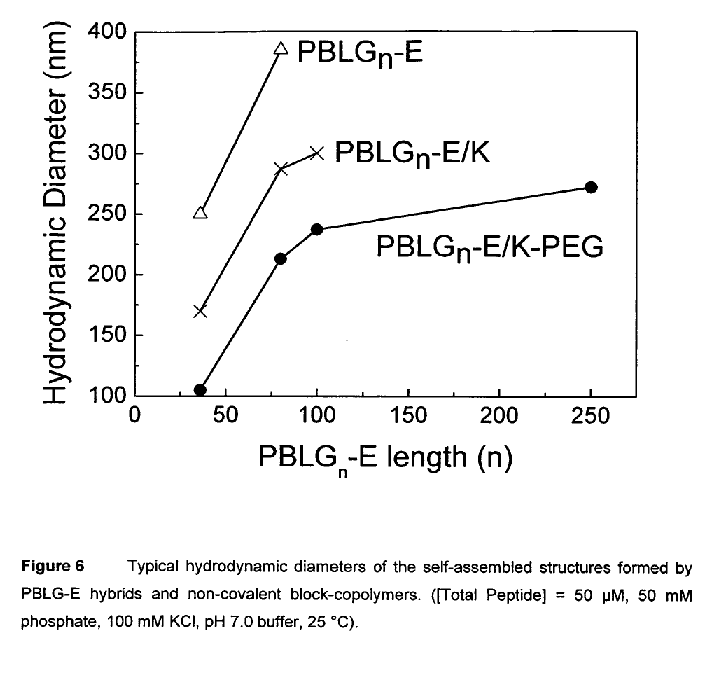

As shown in FIG. 6, the longer the PBLG block, the larger are the particles that form. Additionally, for a particular PBLG block length, the larger the head-group is (through coiled-coil formation of E with K or K-PEG), the smaller the hydrodynamic diameter of the particles. These trends can both be explained by classical packing parameter considerations: the larger the head-group is in comparison to the hydrophobic PBLG, the greater is the curvature of the amphiphile, and hence the particle size decreases (Israelachvili, J. N.; Mitchell, D. J.; Ninham, B. W. Journal of the Chemical Society-Faraday Transactions Ii 1976, 72, 1525-1568). The packing parameter was originally designed to predict the morphology and size of nanostructures formed from lipids, and this approach is not always suited to block copolymers because it does not take into account the complexity of the thermodynamics and interaction free-energies of the blocks (Marsden, H. R et al, Journal of the American Chemical Society 2008, 130, (29), 9386-9393). That being said, it is sufficient to explain the trends observed in the self-assembly of this system. This may be because in the case of both lipid structures and structures formed from the PLBG-E series the influence of stretching of the hydrophobic chains is minimal because the chains do not change their geometry appreciably (lipid tails are stretched (Opsteen, J. A.; Cornelissen, J. J. L. M.; van Hest, J. C. M. Pure and Applied Chemistry 2004, 76, (7-8), 1309-1319, which is incorporated by reference herein), and the PBLG rods have a very well defined structure and size with no change in configuration expected upon aggregation (Halperin, A. Macromolecules 1990, 23, (10), 2724-2731, which is incorporated by reference herein)), so there is no free-energy penalty due to the deformation of the core block.

Particle Morphology: Encapsulation

The average particle sizes determined from DLS indicated that the hybrids and complexes assemble into particles that are larger than micelles. To distinguish between large compound aggregates and vesicles, samples were prepared with the water soluble fluorescent dye Rhodamine B added to the aqueous buffer. Folio wing sonication, the unencapuslated Rhodamine B was removed over a fast protein liquid chromatography (FPLC) column.

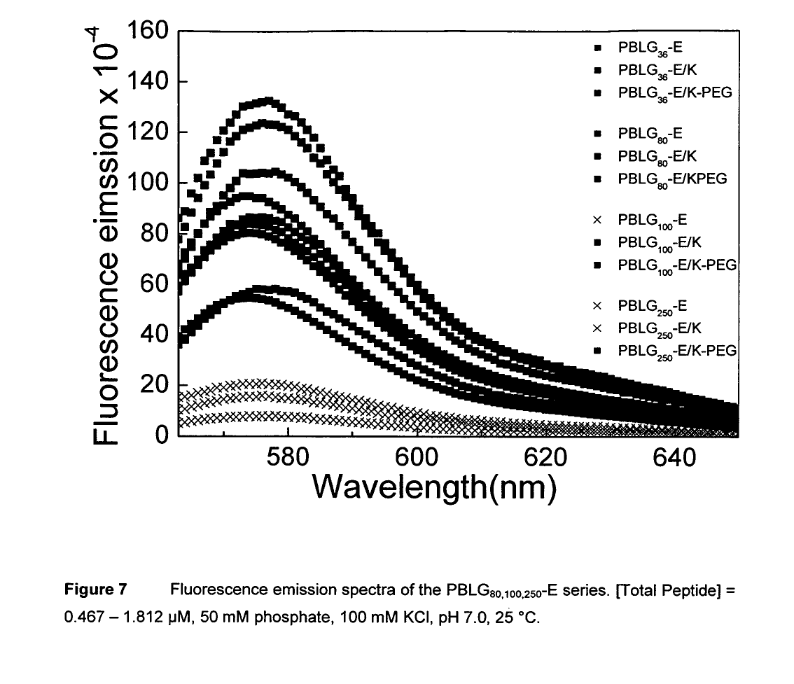

As expected, the samples that did not show well defined self-assembly by DLS contained insignificant amounts of Rhodamine B, as verified by fluorescence spectroscopy. The remainder of the samples exhibited Rhodamine B fluorescence (FIG. 7), indicating that the hybrids and non-covalent complexes had a suitable balance of the hydrophilic block size to the hydrophobic PBLG block to lead to controlled self-assembly, and that these self-assembled structures had aqueous interiors, i.e. were vesicles. These nanocapsules were stable for at least 11 months at 4° C. as determined by DLS.

Particle Morphology: Scanning Electron Microscopy

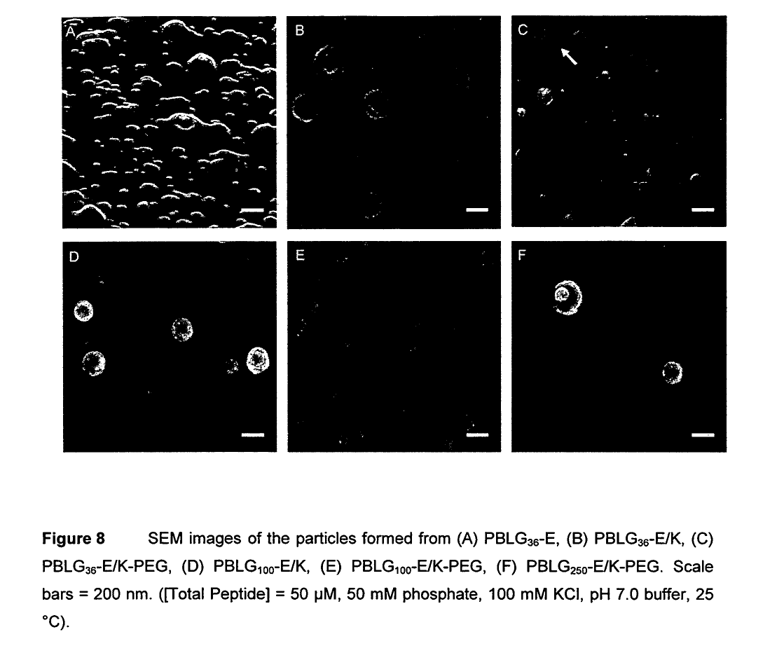

Further information about the morphology of the structures that formed was obtained by scanning electron microscopy of the dispersions (FIG. 8). The effect of PBLG chain length and the combination of the PBLG-E block copolymers with K or K-PEG on the ability of the molecules to controllably self-assemble was the same as observed with DLS. The morphologies of all the ordered structures were circular, being spherical, sunken spherical, or disks. Considering the well-defined lengths of the molecules, the sizes of the particles indicate that there are some spherical micelles, but the majority of the aggregates are larger than this. For PBLG₃₆-E and PBLG₃₆-E/K the sunken spheres suggest that the molecules self-assemble into vesicles in solution, and that the vesicle bilayers are flexible enough to flatten or collapse during drying (FIG. 8A, B). Upon complexation with K-PEG (PBLG₃₆-E/K-PEG) the structures are smaller, as explained in the DLS section, and these smaller spheres are stable upon drying. This sample also contained disk-like aggregates (arrow, FIG. 8C). For the longer PBLG lengths the SEM images exclusively show spherical objects that are unaffected by the drying process, meaning that if they are vesicles their bilayers are rigid enough to withstand the drying process.

Particle Morphology: Cryogenic-Transmission Electron Microscopy

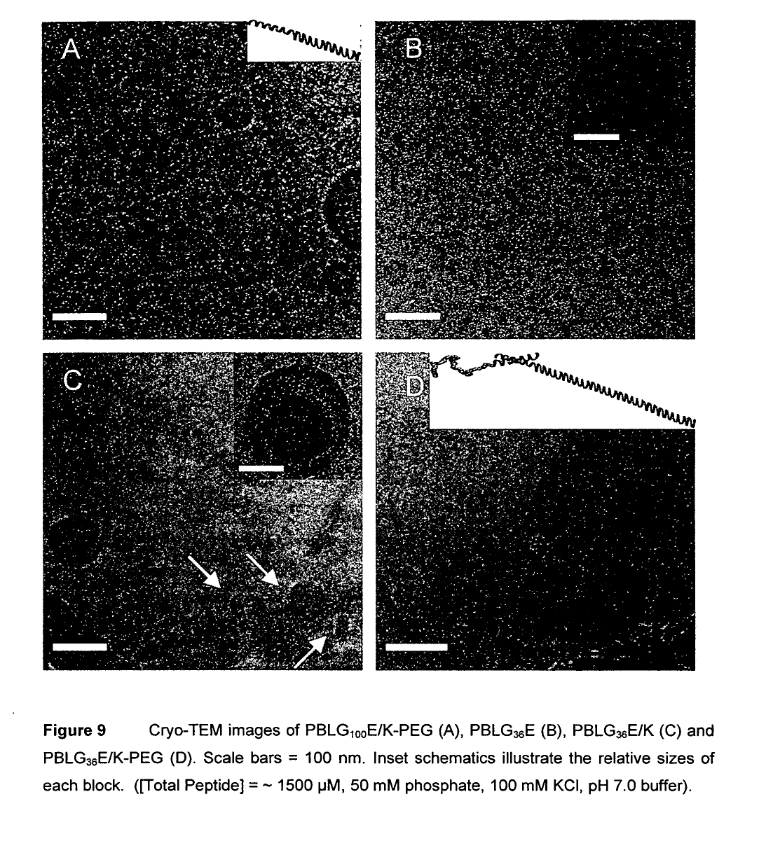

To obtain further insight into the internal structure of the particles cryo-TEM images were obtained for a selection of the self-assembled structures (FIG. 9). These confirm that the preparations, both from the longer and shorter PBLG block lengths, do indeed contain vesicles. Schematics of the molecules are inset into FIG. 9 to give an impression of the relative block lengths of the hybrids/complexes that make up the vesicle bilayers.

The shortest hybrid, PBLG₃₆-E, has a low PDI of 1.1, and self-assembles into vesicles with rather uniform membrane thicknesses, that seem to be independent of the vesicle diameter. The thicknesses observed increases slightly with increasing size of the hydrophilic block/s: 17.2+2.6 nm for PBLG₃₆-E, 18.5+2.4 nm for PBLG₃₆-E/K, and 21.5E/K-PEG +2.2 nm for PBLG₃₆-E/K-PEG (FIG. 9A, B, C). The observed membrane thicknesses are in remarkably close accordance with the calculated bilayer thicknesses, as seen in Table 2.

TABLE 2
Vesicle bilayer thicknesses as measured
with cryo-TEM and calculated.

| Sample | d (nm) cryo-TEM | d (nm) calculated |
|---|---|---|
| PBLG₃₆-E | 17.2 ± 2.6 nm | 18 |
| PBLG₃₆-E/K | 18.5 ± 2.4 nm | 18 |
| PBLG₃₆-E/K-PEG | 21.5 ± 2.2 nm | 23 |
| PBLG₁₀₀-E/K-PEG | 68 ± 22 nm | 42 |

These results show that the rigid hydrophobic PBLG rods can be induced to assemble into very well-defined bilayers through coupling to the water soluble peptide rods. In contrast to other block copolymer vesicles (Srinivas, G.; Discher, D. E.; Klein, M. L. Nature Materials 2004, 3, (9), 638-644, which is incorporated by reference herein), there does not appear to be any interdigitation of the two layers of the hydrophobic block, presumably due to the rod-like structure of the PBLG.

The vesicles composed of PBLG₁₀₀-E/K-PEG have very thick membranes (FIG. 9D). The average membrane thickness is 68 nm, although with quite high variability (std. dev. 22 nm), resulting from the range of PBLG lengths (PDI 1.4). An advantage of the polymersomes of the invention over liposomes is that their membrane thickness varies depending on the composition, molecular weight, and degree of stretching of the blocks. The hydrophobic core of lipid bilayers is always approximately 3-4 nm thick, regardless of the lipid composition (Discher, B. M.; Hammer, D. A.; Bates, F. S.; Discher, D. E. Current Opinion in Colloid & Interface Science 2000, 5, (1-2), 125-131, which is incorporated by reference herein). In the present series the thickness of the membrane can be tuned by the PBLG block length, and it is believed that the thickness of the membrane of PBLG₁₀₀-E/K-PEG vesicles is the largest reported for polymersomes in aqueous solutions.

Although they are of vastly differing sizes, and with different factors influencing their self-assembly to different extents, the majority of natural lipids and polymeric amphiphiles reported so far to form vesicles have a hydrophilic weight or volume fraction is between 20-40% of the total molecular weight or volume (Discher, B. M. et al, Current Opinion in Colloid & Interface Science 2000, 5, (1-2), 125-131).

With the PBLG-E series described herein, vesicles form with as little as 12 hydrophilic weight %, and up to ˜40 hydrophilic weight %, as the phase diagram of Table 3 shows. The ability of the hybrids to assemble in a controlled manner with low hydrophilic block fractions may be because the rod-rod structure of PBLG-E has a strong propensity to form bilayers structures in selective solvents because of the intrinsic orientational order of the rigid rods (Antonietti, M.; Forster, S. Advanced Materials 2003, 15, (16), 1323-1333, which is incorporated by reference herein).

TABLE 3
Hydrophilic weight percent of the hybrids/complexes,
with structural phases indicated.

| | E | K | K-PEG |
|---|---|---|---|
| PBLG₃₆-E | 23 v | 37 v | 51 b, v |
| PBLG₈₀-E | 12 v | 21 v | 32 v |
| PBLG₁₀₀-E | 10 u | 18 v | 27 v |
| PBLG₂₅₀-E | 4 u | 8 u | 13 v |

v denotes vesicles, b denotes bicelles and u undefined aggregation.

The polymersomes of the invention have been investigated for their drug-delivery potential, as they are more robust than the traditional liposome carriers due to their thicker bilayers. Using these polypeptide hybrids the thickness of the membrane, and hence the properties of the polymersomes can be controlled. Another way to control the properties of the PBLGn-E polymersomes is to form the non-covalent coiled-coil complex with K or K-PEG. Coiled-coil formation of E/K-PEG results in vesicles with a PEG corona. PEGylated vesicles are known as ‘stealth’ vehicles, as they have extended circulation times in the body compared to non-PEGylated vesicles (Woodle, M. C. Chemistry and Physics of Lipids 1993, 64, (1-3), 249-262, and Photos, P. J.; Bacakova, L.; Discher, B.; Bates, F. S.; Discher, D. E. Journal of Controlled Release 2003, 90, (3), 323-334, which is incorporated by reference herein). As a wide variety of moieties could be conjugated to K, it is possible to functionalize the surface of the polymersomes in a myriad of ways (for example ligands or antibodies) in order to specify the behavior e.g. targeting of the polymersomes.

The ‘peptosomes’ presented here are analogous to viral capsids: both have self-assembled shells composed of polypeptides, they are robust, they encapsulate molecules, and they include a means for targeting. The targeting can be through the same recognition pattern as viruses—i.e. the coiled-coil interaction, or varied to suit a particular application.

Polymeric Bicelles.

In addition to peptosomes, disks of uniform density are observed in the cryo-TEM images of PBLG₃₆E/K-PEG, as was also observed with SEM (arrows, FIG. 9C). This is the sample with the longest hydrophilic component in comparison to the PBLG block. Presumably polymeric bicelles are observed only for this non-covalent block-copolymer because the length of the PBLG block is short enough that the PEG is able to fold over the exposed PBLG sides of planar bilayers, shielding them from the aqueous buffer. This eliminates the energetic need for the bilayers to close the hydrophobic sides by curving to form vesicles.

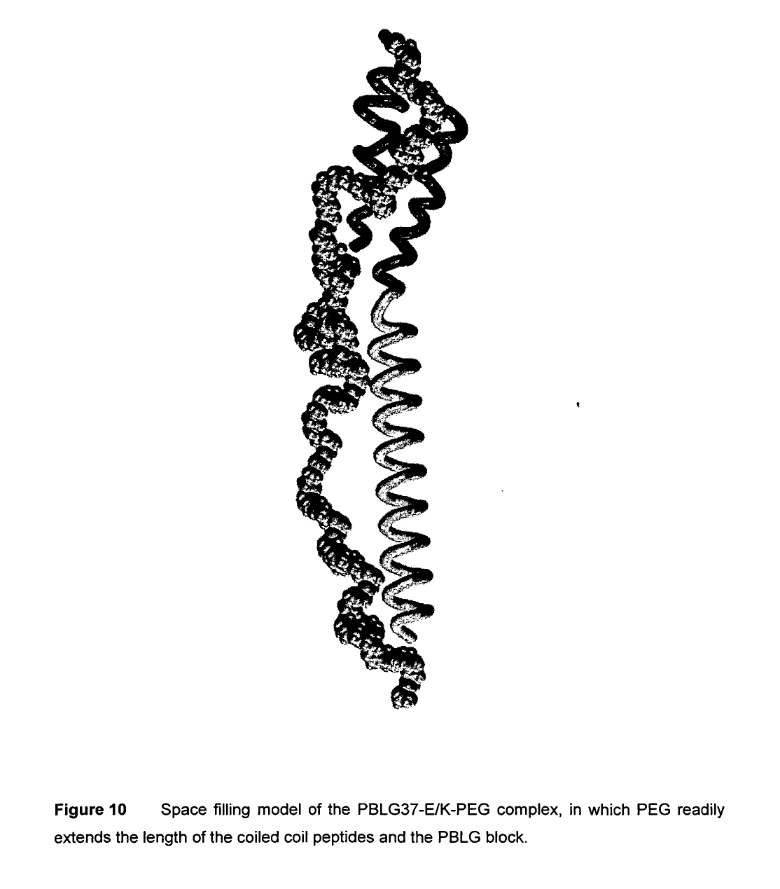

This hypothesis was tested with computer modelling simulations of PBLG37-E/K-PEG using Molden version 4.6 (Noordik, G. S. a. J. H. J. Comput.-Aided Mol. Design 2000, 14, 123-134, which is incorporated by reference herein). The E/K dimer structure is based on the work of Litowski and Hodges (Lindhout, D. A.; Litowski, J. R.; Mercier, P.; Hodges, R. S.; Sykes, B. D. Biopolymers 2004, 75, (5), 367-375, which is incorporated by reference herein). As shown in FIG. 10, PEG is able to cover the length of the PBLG block without any chain stretching, i.e. while still in the random coil configuration.

A theoretical study has found that for rod-coil block copolymers the only stable micellar form has disk-like cores and relatively large corona thicknesses. The disk-like core reduces the core-corona interfacial free energy of the rod blocks, as in this geometry the rods pack well together, and only large coil blocks can deform enough to balance the interfacial free energy (Halperin, A. Macromolecules 1990, 23, (10), 2724-2731).

Experimental Section

Materials

FMOC-protected amino acids were purchased from Novabiochem. Tentagel PAP resin was purchased from Rapp Polymere. Monocarboxy terminated polystyrene was purchased from Polymer Source Inc. All other reagents and solvents were obtained at the highest purity available from Sigma-Aldrich or BioSolve Ltd. and used without further purification.

Solid Phase Peptide Synthesis of the Coiled-Coil Forming Peptides E, K, and K-PEG.

The peptides E and K, and the hybrid K-PEG were prepared and characterized as described in Marsden, H. R. et al, A. Journal of the American Chemical Society 2008, 130, (29), 9386-9393. After the peptide E was prepared, the resin was removed from the reaction vessel, swollen in 1:1 (v/v) DMF:NMP, and FMOC deprotected. The amount of successfully synthesized E on a given weight of peptide-resin was estimated using the mass added to the resin during the synthesis of E, and by integration of HPLC peaks from an LCMS run of a test cleavage of 10 mg of resin-bound peptide.

Synthesis of γ-benzyl L-glutamate N-carboxyanhydride (BLG NCA).

A suspension of γ-benzyl L-glutamate (ca. 5.0 g, 21.1 mmol) in anhydrous ethyl acetate was heated to reflux (120° C.) under an argon atmosphere with vigorous stirring. Triphosgene (ca. 2.1 g, 7.0 mmol) was added quickly and stirring was continued for 3 hours, until the suspension became clear. If the suspension remained turbid a small quantity of triphosgene was added every 15 minutes. The solution was filtered and concentrated to one third of the initial volume (oily yellow liquid). The product was transferred to a glovebox under an argon atmosphere and precipitated in hexane, filtered, recrystallized, and dried. ¹H NMR (300 MHz, CDCl₃, δ): 7.3 (aromatic H, m); 5.1 (benzylic CH2, s), 2.6 (γ-CH2, t), 2.2 (β-CH2, m), 4.4 (α-CH, t), 6.8 (N—H, br).

Solid Phase Synthesis of Poly (γ-benzyl L-glutamate)-block-E (PBLG-E).

Poly(γ-benzyl L-glutamate) was synthesized via a one-pot NCA polymerization of γ-benzyl L-glutamate N-carboxyanhydride, initiated from the amine at the N-terminus of the peptide E while still on the resin. The resin-bound peptide was dried with reduced pressure at 40° C. overnight, and then in argon with reduced pressure for 5 hours. Under an argon atmosphere the peptide-resin was swollen in DCM (2.5 wt % NCA to DCM), and subsequently the appropriate weight of NCA (determined from the mass loading and HPLC peak integration) was added. The flask was shaken for 24-65 hrs. A small volume of DCM was drained from the reaction vessel and the contents analyzed with FT-IR spectroscopy, showing that no NCA monomer remained (absence of the carbonyl stretching absorption band of C₂ at 2000-1800 cm⁻¹, which is released as CO₂ during the reaction). The resin was drained and washed profusely with DCM, NMP, DMF, and finally with DCM. The initial DCM washes were dried to collect any homopolymer that formed in solution. The yields of the resin-bound block copolypeptides were 85% -92%.

The hybrid material was cleaved in the protected form from the resin using 1:99 (v/v) TFA:DCM for 2 minutes, 10 times. Each cleavage mixture was precipitated drop-wise in cold methanol. The white precipitate was compacted with centrifugation and the supernatant removed. This was repeated three times with the addition of fresh methanol. The pellets were vacuum-dried.

The O-t-Bu and BOC protecting groups of the glutamic acid and lysine residues of the E block were removed by stirring the hybrid in 47.5:47.5:2.5:2.5 (v/v) TFA:DCM:water:TIS for 1 hour, and the product was precipitated drop-wise in cold methanol. The white precipitate was compacted with centrifugation and the supernatant removed. This was repeated three times with the addition of fresh methanol. The pellets were vacuum-dried, with yields ranging from 28-74% (Table 1).

Characterization of the PBLG-E Block Copolymers.

Molecular weights and their distributions of the protected PBLG-E hybrids was determined using gel phase chromatography (GPC). GPC was performed with a Shimadzu system equipped with a refractive index detector. A Polymer Laboratories column was used (3M-RESI-001-74, 7.5 mm diameter, 300 mm length) with DMF as the eluent, at 60° C., and a flow rate of 1 mL min⁻¹. Both the coiled-coil peptide and PBLG are soluble in DMF, and the runs were conducted at 60° C. to prevent aggregation. The molecular weights were calibrated using polystyrene standards.

The purity and molecular weights of the deprotected hybrids were checked using ¹H-NMR spectra recorded on a Bruker AV-500 spectrometer and a Bruker DPX300 spectrometer at room temperature. The residual proton resonance of deuterated dichloromethane was used for calibration. A range of ¹H-NMR spectra of the deprotected hybrids were recorded, from deuterated dichloromethane to 1:1 (v/v) deuterated dichloromethane:trifluoroacetic acid.

The absolute masses of the hybrids with shorter PBLG blocks could be determined using MALDI-TOF mass spectrometry. Spectra were acquired using an Applied Biosystems Voyager System 6069 MALDI-TOF spectrometer. Samples were dissolved in 1:1 (v/v) 0.1% TFA in water:acetonitrile (TA), at concentrations of ˜3 mg mL⁻¹. Solutions for spots consisted of (v/v) 1:10 sample solution: 10 mg mL⁻¹ACH in TA.

The secondary structure of the block copolymers was determined using FT-IR spectroscopy. FT-IR spectra were recorded on a BIORAD FTS-60A instrument equipped with a deuterated-triglycine-sulphate (DTGS) detector at a resolution of 20 cm-1. The compounds were dried from dichloromethane onto an ATR ZnSe crystal. A blank ATR ZnSe crystal was used as the background.

Preparation of PBLG-E Suspensions.

0.1 μmol of each compound (PBLG-E, or PBLG-E and K, or PBLG-E and K-PEG) were dissolved in 200 μL tetrahydrofuran (THF). 2 mL phosphate buffered saline (PBS, 50 mM PO4, 100 mM KCl, pH 7.0) was added and the sample immediately sonicated for 2 hours in a Branson 1510 bath sonicator with an output of 70 W and 42 kHz. The final concentration of each molecule was 50 μM.

For the encapsulation of Rhodamine B in the vesicles the samples were prepared as described above, with the addition of Rhodamine B (0.2 mg mL⁻¹, 0.418 mM) to the buffer. The unencapsulated Rhodamine B was removed over a fast protein liquid chromatography (FPLC) column.

Characterization of PBLG-E Suspensions.

Experimental diffusion coefficients, D, were measured at 25° C. by dynamic light scattering (DLS) using a Malvern Zetasizer Nano ZS equipped with a peltier-controlled thermostatic cell holder. The laser wavelength was 633 nm and the scattering angle was 173°. The Stokes-Einstein relationship D=k_{b}T/3πηDₙ was used to estimate the hydrodynamic radius, Dₙ. Here k_{b} is the Boltzman constant, and η is the solvent viscosity.

Scanning electron microscopy (SEM) was conducted on a Nova NanoSEM FEI instrument with an accelerating voltage of 10 kV and spot size of 3.5. Samples for SEM were prepared by placing 5 μL of the solution on SEM stubs with a TEM grid on the carbon tape. After 30 minutes the excess buffer was removed. Samples were coated with gold for one minute, resulting in a layer ˜15 nm thick.

Transmission electron microscopy (TEM) was conducted on a JEOL 1010 instrument with an accelerating voltage of 60 kV. Samples for TEM were prepared by placing a drop of each solution on carbon-coated copper grids. After ˜10 minutes the droplet was removed from the edge of the grid. A drop of 2% PTA stain was applied and removed after 2 minutes. Negative images are shown in order to retain image quality.

Samples for cryogenic TEM were concentrated by centrifuging in Centricon centrifugal filter devices MWCO 3000 g mL-1 at 4° C. Sample stability was verified by DLS and TEM. The cryogenic transmission microscopy measurements were performed on a FEI Technai 20 (type Sphera) TEM or on a Titan Krios (FEI). A Gatan cryo-holder operating at ˜−170° C. was used for the cryo-TEM measurements. The Technai 20 is equipped with a LaB₆ filament operating at 200 kV and the images were recorded using a 1 k×1 k Gatan CCD camera. The Titan Krios is equipped with a field emission gun (FEG) operating at 300 kV. Images were recorded using a 2 k×2 k Gatan CCD camera equipped with a post column Gatan energy filter (GIF). The sample vitrification procedure was carried out using an automated vitrification robot: a FEI Vitrobotä Mark III. TEM grids, both 200 mesh carbon coated copper grids and R2/2 Quantifoil Jena grids were purchased from Aurion. Copper grids bearing lacey carbon films were home made using 200 mesh copper grids from Aurion. Grids were treated with a surface plasma treatment using a Cressington 208 carbon coater operating at 25 A for 40 seconds prior to the vitrification procedure.

Circular Dichroism (CD) spectra were obtained using a Jasco J-815 spectropolarimeter equipped with a peltier-controlled thermostatic cell holder. Spectra were recorded from 260 nm to 200 nm in a 1 mm quartz cuvette at 25° C. Data was collected at 0.5 nm intervals with a 1 nm bandwidth and 1 s readings. Each spectrum was the average of 5 scans. For analysis each spectrum had the appropriate background spectrum (buffer or buffer/THF) subtracted.

FPLC was performed with an Äkta prime, Amarsham Pharmacia Biotech apparatus with a Pharmacia XK 26 column (135 mm×25 mm) packed with Sephadex G50-fine. PBS was used as the eluent. The flow rate was 5 mL min⁻¹, UV sensitivity was set on 0.1 AU, 1%, the conductivity was set on 15-20 mS cm⁻¹ and the wavelength for UV recording was 254 nm. The amount of encapsulated Rhodamine B in each sample was determined by fluorescence spectroscopy, with excitation at 555 nm, and emission monitored from 563-650 nm with 5 nm slits using a Cary-50 Spectrophotometer.

The scope of the invention is defined by the following claims.

1. A block copolypeptide comprising a hydrophilic heteropolypeptide block (A) and a hydrophobic homopolypeptide block (B).

2. The block copolypeptide of claim 1 wherein block (A) is capable of forming a coiled coil complex with a complementary peptide.

3. The block copolypeptide of claim 2 wherein block (A) comprises from 2 to about 200 heptad units and wherein block (A) is capable of forming a left-handed coiled coil with a complementary peptide.

4. The block copolypeptide of claim 3 wherein block (A) comprises (E IA ALE K)ₙ₁ wherein n=from about 3 to about 10.

5. The block copolypeptide of claim 1 wherein the hydrophobic homopolypeptide block (B) is capable of self-assembling into a three-dimensional configuration.

6. The block copolypeptide of claim 5 wherein the three-dimensional configuration is an α-helix or a β-sheet.

7. The block copolypeptide of claim 1 wherein the hydrophobic homopolypeptide block (B) comprises from about 10 to about 1000 amino acid residues.

8. The block copolypeptide claim 1 wherein the hydrophobic homopolypeptide block (B) is a homopolyamino acid wherein the amino acid is hydrophilic, and wherein a polar group of the amino acid is protected by a hydrophobic protecting group to render the homopolyamino acid hydrophobic.

9. The block copolypeptide of claim 8 wherein block (B) is poly(γ-benzyl L-glutamate) (PBLG).

10. A process for preparing a block copolypeptide of claim 1 comprising the steps of:
(a) preparing a hydrophilic heteropolypeptide block (A);
(b) preparing a hydrophobic homopolypeptide block (B); and
(c) covalently attaching block (A) to block (B) to form the block copolypeptide.

11. The process of claim 10 wherein step (a) comprises solid phase synthesis of the heteropolypeptide block (A).

12. The process of claim 10 wherein step (b) comprises ring-opening polymerisation (ROP) of an amino acid N-carboxyanhydride (NCA) to form the homopolypeptide block (B).

13. The process of claim 12 wherein the ROP of the NCA is initiated from the heteropolypeptide block (A).

14. A method for preparing a copolymer comprising ring-opening polymerisation (ROP) of an amino acid N-carboxyanhydride (NCA) initiated from a peptide.

15. The method of claim 14 comprising the steps of (i) solid phase synthesis of the peptide.

16. The method claim 14 wherein the ROP of the NCA is initiated from the peptide on a solid support.

17. A polymersome comprising a block copolypeptide of claim 1.

18. The polymersome of claim 17 further comprising a complementary peptide.

19. The polymersome of claim 18 wherein the complementary peptide further comprises a functional group.

20. The polymersome of claim 19 wherein the complementary peptide is a peptide-poly(ethylene glycol) hybrid.

21. A process for preparing a polymersome of claim 17 comprising mixing the block copolypeptide, and optionally a complementary peptide, in a suitable solvent to form the polymersome.

22. The process of claim 21, comprising a sonication step.

23. (canceled)

24. A drug delivery device comprising a block copolypeptide, wherein the block copolypeptide comprises a hydrophilic heteropolypeptide block (A) and a hydrophobic homopolypeptide block (B).

25-33. (canceled)

34. A composition comprising:
(a) a polymersome comprising a block copolypeptide, wherein the block copolypeptide comprises a hydrophilic heteropolypeptide block (A) and a hydrophobic homopolypeptide block (B); and
(b) a drug encapsulated in the polymersome.

35. The composition of claim 34, wherein the drug is a vaccine.

36. The composition of claim 35, wherein the vaccine is an influenza vaccine.

37. The composition of claim 34, further comprising polyethylene glycol (PEG).

38. The composition of claim 34, further comprising a ligand or an antibody conjugated to the polymersome.
